(19) United States
(12) Patent Application Publication
(10) Pub. No.: US 2010/0055067 A1
(43) Pub. Date: Mar. 4, 2010

(54) PERFLUOROCARBON CONJUGATE AS A BLOOD SUBSTITUTE

(75) Inventor: Seung Bum Park, Seoul (KR)

Correspondence Address: Seung Bum Park, 122-I-502 Seoul National University Faculty Apt., Bongchun-dong, Gwanak-gu, Seoul, KR

(73) Assignee: SNU R&DB Foundation

(21) Appl. No.: 12/198,751

(22) Filed: Aug. 26, 2008

(51) Int. Cl. A61K 31/77; C08G 65/32; A61P 7/00; B32B 5/16

(52) U.S. Cl. 424/78.17; 525/50; 428/402; 525/403

(57) ABSTRACT

There are provided disclosures relating to a conjugate of a perfluorocarbon compound and a cationic polymer wherein the conjugate is a blood substitute.

### BACKGROUND

The use of blood substitutes in the medical field is widely practiced. Examples of two “blood substitute” categories are volume expanders and oxygen therapeutics. Volume expanders are inert and merely increase blood volume. Oxygen therapeutics mimic mammalian blood's oxygen transport ability. Oxygen therapeutics can be divided in two categories based on transport mechanism: perfluorocarbon based, and hemoglobin based (Squires J. E. Science (2002) 295:1002-1005 and Inayat et al., Transfusion and Apheresis Science (2006) 34:25-32). Perfluorocarbons are aliphatic molecules that possess strong intramolecular bonding which helps in preventing their degradation in the blood stream. Perfluorocyclocarbon liquids and emulsions containing particles of these perfluorocarbons have been shown to be useful as artificial bloods and perfusates for organs (Clark, U.S. Pat. No. 3,911,138). Emulsions containing emulsified particles of perfluorocyclocarbons have been prepared (Yokoyama et al., U.S. Pat. No. 3,962,439).

### SUMMARY

Conjugate compositions comprising a perfluorocarbon compound and a cationic polymer wherein the conjugate is a blood substitute and methods for their use are provided herein. The conjugation of the perfluorocarbon compound with the cationic polymer results in enhanced solubility in aqueous medium as compared to an unconjugated perfluocarbon compound and enhanced oxygen absorbing ability as compared to an unconjugated perfluocarbon compound.

In one embodiment, there is provided a conjugate comprising a perfluorocarbon compound and a cationic polymer wherein the conjugate is a blood substitute. In some embodiments, the perfluorocarbon compound is an oxygen-transferable saturated perfluorocarbon. In some embodiments, the perfluorocarbon compound comprises 8 to 25 carbon atoms. In some embodiments, the perfluorocarbon compound is perfluorodecalin, perfluoro(methyldecalin), perfluorooctyl bromide, or dodecafluoropentane.

In some embodiments, the perfluorocarbon compound is a perfluorocycloalkane or a perfluoro(alkylcycloalkane). In some embodiments, the perfluoro(alkylcycloalkane) is selected from the group consisting of perfluoro(methylpropylcyclohexanes), perfluoro(butylcyclohexanes), perfluoro(trimethylcyclohexanes), perfluoro(ethylpropylcyclohexanes) and perfluoro (pentylcyclohexanes).

In some embodiments, the perfluorocarbon compound is a perfluoro(alkyltetrahydropyrans). In some embodiments, the perfluoro(alkyltetrahydropyrans) is selected from the group consisting of perfluoro(butyltetrahydropyrans), perfluoro(pentyltetrahydropyrans) and perfluoro(hexyltetrahydropyrans).

In some embodiments, the perfluorocarbon compound is a perfluoro(alkyltetrahydrofurans). In some embodiments, the perfluoro(alkyltetrahydrofurans) is selected from the group consisting of perfluoro(pentyltetrahydrofurans), perfluoro(hexyltetrahydrofurans) and perfluoro(heptyltetrahydrofurans).

In some embodiments, the perfluorocarbon compound is a perfluoro(N-alkylpiperidines). In some embodiments, the perfluoro(N-alkylpiperidines) is selected from the group consisting of perfluoro(N-pentylpiperidines), perfluoro(N-hexylpiperidines) and perfluoro (N-butylpiperidine).

In some embodiments, the perfluorocarbon compound is a perfluoro(N-alkylmorpholines). In some embodiments, the perfluoro(N-alkylmorpholines) is selected from the group consisting of perfluoro(N-pentylmorpholines), perfluoro(N-hexylmorpholines) and perfluoro(N-heptylmorpholines).

In some embodiments, the perfluorocarbon compound is a perfluoro(tert-amine). In some embodiments, the perfluoro(tert-amine) is selected from the group consisting of perfluoro(diethylhexylamines), perfluoro(dipropylbutylamines) and perfluoro (diethylcyclohexyl amines).

In some embodiments, the perfluorocarbon compound is a perfluoro(dioxa-alkane). In some embodiments, the perfluoro(dioxa-alkane) is selected from the group consisting of perfluoro(tetramethylene glycol diisoproyl ether), perfluoro(trimethylene glycol diisopropyl ether), perfluoro(trimethylene glycol diisobutyl ether), and perfluoro(isopropylidene glycol di-n-propyl ether).

In some embodiments, the conjugate is a nanoparticle.

In some embodiments, a particle size of the conjugate is 10 nm (nanometer) to 800 nm. In some embodiments, the particle size of the conjugate is 50 nm to 250 nm.

In some embodiments, the conjugate is soluble in aqueous medium.

In some embodiments, the conjugate is administered to a mammal.

In some embodiments, the cationic polymer is a polyethylene-glycol based cationic hyperbranched polymer. In some embodiments, the cationic polymer is an amine-modified polyethylene-glycol based cationic hyperbranched polymer.

In some embodiments, the conjugate supplements blood of a mammal.

In some embodiments, the conjugate transports oxygen with a p50 of about 10 mmHg to about 50 mmHg.

In some embodiments, the conjugate transports oxygen of about 10% to 50% by volume.

In some embodiments, the perfluorocarbon compound and the cationic polymer are conjugated by a covalent bond.

In some embodiments, the conjugate has an enhanced solubility in aqueous medium as compared to an unconjugated perfluocarbon compound.

In some embodiments, the conjugate has an enhanced oxygen absorbing ability as compared to an unconjugated perfluocarbon compound.

In another aspect, there is provided a pharmaceutical composition comprising the conjugate and a pharmaceutically acceptable carrier. In some embodiments, there is provided a container containing the pharmaceutical composition.

In yet another aspect, there is provided a process of preparation of a conjugate of a perfluorocarbon compound and a cationic polymer wherein the conjugate is a blood substitute, the process comprising reacting the perfluorocarbon compound with the cationic polymer in presence of a base.

In some embodiments, the base is an organic or an inorganic base. In some embodiments, the base is selected from the group consisting of a tertiary amine, a carbonate, and a silicate of sodium or potassium.

In yet another aspect, there is provided a method of making a conjugate of a perfluorocarbon compound and a cationic polymer wherein the conjugate is a blood substitute comprising, reacting the perfluorocarbon compound with the cationic polymer thereby resulting in the conjugate. In some embodiments, the reaction is carried out in presence of a base. In some embodiments, the base is an organic or an inorganic base.

In yet another aspect, there is provided a method of supplementing a blood of a mammal comprising administering to the mammal a composition comprising a conjugate of a perfluorocarbon compound and a cationic polymer wherein the conjugate is a blood substitute and a pharmaceutically acceptable carrier.

In some embodiments, the administering is by an implant, injection or transfusion.

In some embodiments, the mammal suffers from anemia, anemia related conditions, hypoxia or ischemia. In some embodiments, the mammal needs blood transfusion. In some embodiments, the mammal is in trauma.

The foregoing summary is illustrative only and is not intended to be in any way limiting. In addition to the illustrative aspects, embodiments, and features described above, further aspects, embodiments, and features will become apparent by reference to the drawings and the following detailed description.

### DETAILED DESCRIPTION OF THE PRESENT TECHNOLOGY

The illustrative embodiments described in the detailed description and claims are not meant to be limiting. Other embodiments may be utilized, and other changes may be made, without departing from the spirit or scope of the subject matter presented here.

It must be noted that as used herein, and in the appended claims, the singular forms “a,” “an,” and “the” include plural references unless the context clearly dictates otherwise.

Unless defined otherwise, all technical, and scientific terms used herein have the same meanings as commonly understood by one of ordinary skill in the art to which the present technology belongs. Although any methods and materials similar or equivalent to those described herein can be used in the practice or testing of the present technology, illustrative methods, devices, and materials have been described. All publications cited herein are incorporated herein by reference in their entirety for the purpose of describing and disclosing the methodologies, reagents, and tools reported in the publications that might be used in connection with the present technology. Nothing herein is to be construed as an admission that the present technology is not entitled to antedate such publications.

The practice of the present technology will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag).

The terms “alkyl” or “alkane,” refer to monovalent saturated aliphatic hydrocarbyl groups having from 1 to 10 carbon atoms and, in some embodiments, from 1 to 6 carbon atoms. This term includes, for example, linear and branched hydrocarbyl groups such as methyl (CH₃), ethyl (CH₃CH₂), n-propyl (CH₃CH₂CH₂), isopropyl ((CH₃)₂CH), n-butyl (CH₃CH₂CH₂CH₂), isobutyl ((CH₃)₂CHCH₂), sec-butyl ((CH₃)(CH₃CH₂)CH), t-butyl ((CH₃)₃C), n-pentyl (CH₃CH₂CH₂CH₂CH₂), neopentyl ((CH₃)₃CCH₂), and octyl (CH₃(CH₂)₇). A prefix indicating the number of carbon atoms (e.g., C₃-C₅) refers to the total number of carbon atoms in the alkyl group.

The term “alkylene,” refers to divalent saturated aliphatic hydrocarbyl groups having from 1 to 10 carbon atoms and, in some embodiments, from 1 to 6 carbon atoms. The alkylidene and alkylene groups include branched and straight chain hydrocarbyl groups. For example, methylene, ethylene, propylene, isopropylene, pentylene, and the like.

The term “alicyclic,” refers to an organic compound that is both aliphatic and cyclic. The alicyclic compounds contain one or more all-carbon rings which may be either saturated or unsaturated. Examples of alicyclic compounds include, but are not limited to, cycloalkanes, cyclopropane, cyclobutane, and cyclohexane, etc.

The terms “cycloalkane” or “cycloalkyl,” refer to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkane” applies when the point of attachment is at a non-aromatic carbon atom (e.g., 5,6,7,8,-tetrahydronaphthalene-5-yl). The term “cycloalkane” includes cycloalkenyl groups. Examples of cycloalkyl groups include, but are not limited to, cyclopropyl, cyclobutyl, cyclopentyl, cyclohexyl, cyclooctyl, and cyclohexenyl. A polycyclic cycloalkane is decalin. Decalin also known as, decahydronaphthalene or bicyclo[4.4.0]decane, is a bicyclic compound. Decalin can be in cis or trans form.

The term “alkylcycloalkane” refers to the cycloalkane substituted with the alkyl.

The terms “dioxalkane” or “alkylene glycol dialkyl ether” is used interchangeably herein. For example, the structure of perfluoro(tetramethylene glycol diisopropyl ether) is:

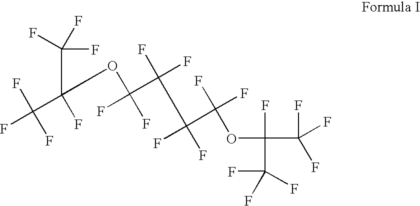

Examples of dioxalkane include, but are not limited to, perfluoro(3,8-dioxa-2,9-dimethyldecane) or perfluoro(tetramethylene glycol diisopropyl ether), perfluoro(3,7-dioxa-2,8-dimethylnonane) or perfluoro (trimethylene glycol diisopropyl ether) and perfluoro (4,6-dioxa-5,5-dimethylnonane) or perfluoro(isopropylene glycol di-n-propyl ether).

The term “heterocyclic,” refers to a saturated or partially saturated cyclic group having from 1 to 14 carbon atoms and from 1 to 6 heteroatoms selected from the group consisting of nitrogen, sulfur, or oxygen and includes single ring and multiple ring systems including, but not limited to, fused, bridged, and spiro ring systems. For multiple ring systems having aromatic and/or non-aromatic rings, the terms “heterocyclic” apply when there is at least one ring heteroatom and the point of attachment is at an atom of a non-aromatic (e.g., 1,2,3,4-tetrahydroquinoline-3-yl, 5,6,7,8-tetrahydroquinoline-6-yl, and decahydroquinolin-6-yl). More specifically the heterocyclyl includes, but is not limited to, tetrahydropyranyl, tetrahydrofuranyl, piperidinyl, N-alkylpiperidinyl, piperazinyl, N-methylpyrrolidin-3-yl, 3-pyrrolidinyl, 2-pyrrolidon-1-yl, morpholinyl, and pyrrolidinyl.

The term “alkyltetrahydropyran” refers to tetrahydropyran substituted with the alkyl.

The term “N-alkylpiperidine” refers to piperidine substituted with an alkyl at the nitrogen of the piperidine.

The term “N-alkylmorpholine” refers to morpholine substituted with an alkyl at the nitrogen of the morpholine.

The term “aqueous medium” refers to a medium comprising at least 60% of water.

The term “carbonate” refers to a salt of carbonic acid where the carbonate ion CO₃²⁻ is attached to two positively charged ions. Examples of the positively charged ion include, but are not limited to, potassium, sodium, ammonium, calcium, etc.

The term “bicarbonate” refers to a bicarbonate ion HCO₃⁻ attached to one positively charged ion. Examples of the positively charged ion include, but are not limited to, potassium, sodium, ammonium, calcium, etc.

The term “silicate” refers to a silicate ion SiO₃²⁻ attached to two positively charged ions. Examples of the positively charged ion include, but are not limited to, potassium, sodium, ammonium, calcium, magnesium, etc.

The term “tert-amine,” refers to a tertiary amine —N(R)₃ where R is alkyl or cycloalkane. For example, diethylhexylamine, dipropylbutylamine and diethylcyclohexylamine, among others.

### A. Compositions

In one embodiment, there is provided a conjugate comprising a perfluorocarbon compound and a cationic polymer wherein the conjugate is a blood substitute. “Perfluorocarbon” as used herein, means an organic compound in which all hydrogens have been replaced with fluorine. By way of example only, a perfluorocycloalkane is a cycloalkane where all the hydrogens have been replaced with fluorine; a perfluoro(alkylcycloalkane) is an alkylcycloalkane where all the hydrogens have been replaced with fluorine; a perfluoro(alkyltetrahydropyran) is an alkyltetrahydropyran where all the hydrogens have been replaced with fluorine; a perfluoro(N-alkylpiperidine) is a N-alkylpiperidine where all the hydrogens have been replaced with fluorine; and a perfluoro(N-alkylmorpholine) is a N-alkylmorpholine where all the hydrogens have been replaced with fluorine.

Perfluorocarbon compounds are not miscible in water and hence cannot transport water soluble metabolites or waste products in the circulation, which may limit their ability to sustain life over long periods. Perfluorocarbons are not miscible with aqueous solutions and are prepared as emulsions before they are used as blood substitutes. The reticulo-endothelial system can systemically remove perfluorocarbons that are finally exhaled via the alveolar surfaces in the lungs resulting in a short dose dependent circulatory half-life.

In cell-free hemoglobin solutions, oxygen is bound to hemoglobin in the same way as it is bound to the native molecule. However, in perfluorocarbons, the oxygen readily dissolves in the chemically inert perfluorocarbon liquid and can be easily extracted by oxygen-deprived tissues. The oxygen loading capacity of perfluorocarbons is linearly related to the partial pressure of oxygen in equilibrium with the emulsion. This linear oxygen binding relationship can result in narrow physiological P⁰2 which can raise possible toxicities issues related to prolonged exposure to high concentrations of oxygen.

The fluorocarbon FC-43 (Trade Mark of perfluorotributylamine sold by Minnesota Mining and Manufacturing Co., St. Paul, Minn.) and Freon E-4 (Trade Mark of 2-monohydrononacosafluoro-3,6,9,1 2-tetraoxa-5,8,11-trimethylpentadecan sold by DuPont de Nemours & Company, Wilmington, Del.) are known to accumulate in the internal organs such as liver and spleen for a long period of time when administered to test animals, resulting in an adverse effect to the animals. The fluorocarbon FX-80 (Trade Mark of perfluorotetrahydrofuran supplied by Minnesota Mining and Manufacturing Co., St. Paul, Minn.) has a relatively low boiling point and can cause substantial damage to the lung.

It is contemplated that the conjugate comprising a perfluorocarbon compound and a cationic polymer eliminates or reduces such adverse effects to the organs or tissues. The conjugation of the perfluorocarbon compound with the cationic polymer results in enhanced solubility in aqueous medium as compared to an unconjugated perfluocarbon compound and enhanced oxygen absorbing ability as compared to an unconjugated perfluocarbon compound. The composition comprising the conjugate of the prefluorocarbon compound and the cationic polymer can pass through the narrowed blood vessel due to its small size and high solubility.

In some embodiments, the conjugate comprises a saturated perfluorocarbon compound comprising 8 to 25 carbon atoms some or all of which form at least one of a saturated alicyclic ring, a heterocyclic ring together with hetero nitrogen atom and/or oxygen atom, an aliphatic tertiary amine together with nitrogen atom or an aliphatic ether together with oxygen atom or atoms.

In some embodiments, the perfluorocarbon compound is perfluorodecalin, perfluoro(methyldecalin), perfluorooctyl bromide, or dodecafluoropentane.

In some embodiments, the perfluorocarbon compound is a perfluorocycloalkane or perfluoro(alkylcycloalkane) which includes, but is not limited to, perfluoro (C₃₋₅-alkylcyclohexanes) such as perfluoro(methylpropylcyclohexanes), perfluoro (butylcyclohexanes), perfluoro(trimethylcyclohexanes), perfluoro(ethylpropylcyclohexanes) and perfluoro (pentylcyclohexanes); perfluorodecalin and perfluoro (methyldecalines).

In some embodiments, the perfluorocarbon compound is a perfluoro(alkylsaturated-heterocyclic compound) which includes, but is not limited to, perfluoro(alkyltetrahydropyrans), perfluoro(alkyltetrahydrofurans), perfluoro (N-alkylpiperidines), and perfluoro(N-pentylmorpholines). The examples of perfluoro(alkyltetrahydropyrans) include, but are not limited to, perfluoro(butyltetrahydropyrans), perfluoro(pentyltetrahydropyrans) and perfluoro(hexyltetrahydropyrans). The examples of perfluoro(alkyltetrahydrofurans) include, but are not limited to, perfluoro (pentyltetrahydrofurans), perfluoro(hexyltetrahydrofurans) and perfluoro(heptyltetrahydrofurans). The examples of perfluoro (N-alkylpiperidines) include, but are not limited to, perfluoro(N-pentylpiperidines), perfluoro(N-hexylpiperidines) and perfluoro (N-butylpiperidine). The examples of perfluoro(N-alkylmorpholines) include, but are not limited to, perfluoro(N-pentylmorpholines), perfluoro(N-hexylmorpholines) and perfluoro(N-heptylmorpholines).

In some embodiments, the perfluorocarbon compound is a perfluoro(tert-amine) which includes, but is not limited to, perfluoro(diethylhexylamines), perfluoro(dipropylbutylamines) and perfluoro (diethylcyclohexyl amines).

In some embodiments, the perfluorocarbon compound is a perfluoro(dioxalkane), or perfluoro(alkylene glycol dialkyl ether). The examples include, but are not limited to, perfluoro(3,8-dioxa-2,9-dimethyldecane) or perfluoro(tetramethylene glycol diisopropyl ether), perfluoro(3,7-dioxa-2,8-dimethylnonane) or perfluoro (trimethylene glycol diisopropyl ether) and perfluoro (4,6-dioxa-5,5-dimethylnonane) or perfluoro(isopropylene glycol di-n-propyl ether).

These perfluorocarbon compounds can be used alone or in a mixture of two or more kinds of the compounds.

In some embodiments, the perfluorocarbon compounds include but are not limited to, perfluorodecalin and perfluoro(methyldecalin). These perfluorocarbon compounds have faster excretion from the body.

In some embodiments, the cationic polymer is a polyethylene-glycol based cationic hyperbranched polymer. In some embodiments, the cationic polymer is an amine-modified polyethylene-glycol based cationic hyperbranched polymer. In some embodiments, the cationic polymer is an amine-modified polyethylene-glycol based cationic hyperbranched polymer as shown in Formula II below (Khan et al., Biomacromolecules (2006) 7:1386-1388 and its Supporting Information are both incorporated herein by reference in their entirety).

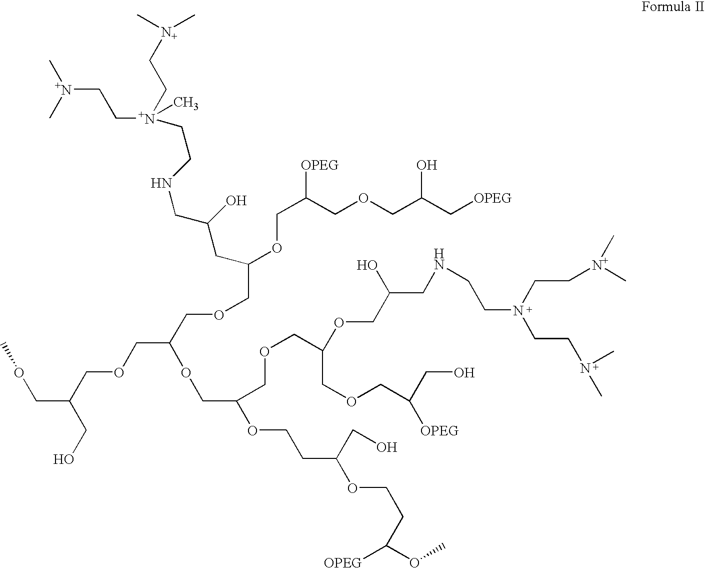

In some embodiments, the cationic polymer is an amine-modified polyacrylamide based cationic hyperbranched polymer. In some embodiments, the cationic polymer is an amine-modified PEI (polyethylenimine) based cationic hyperbranched polymer. In some embodiments, the cationic polymer is an amine-modified cationic PAMAM (polyamidoamine) dendrimer. By way of example only, the amine-modified cationic PAMAM (polyamidoamine) dendrimer is as shown in Formula III. In some embodiments, the cationic polymer is a copolymer of any of the above-mentioned cationic polymers. It is to be understood that a molecular weight of the cationic polymer may be selected by one of ordinary skill in the art to provide for a composition having a viscosity that is similar to that of the host's (or mammal's) blood. In some embodiments, the viscosity may be slightly lower or slightly higher than the viscosity of the host's blood.

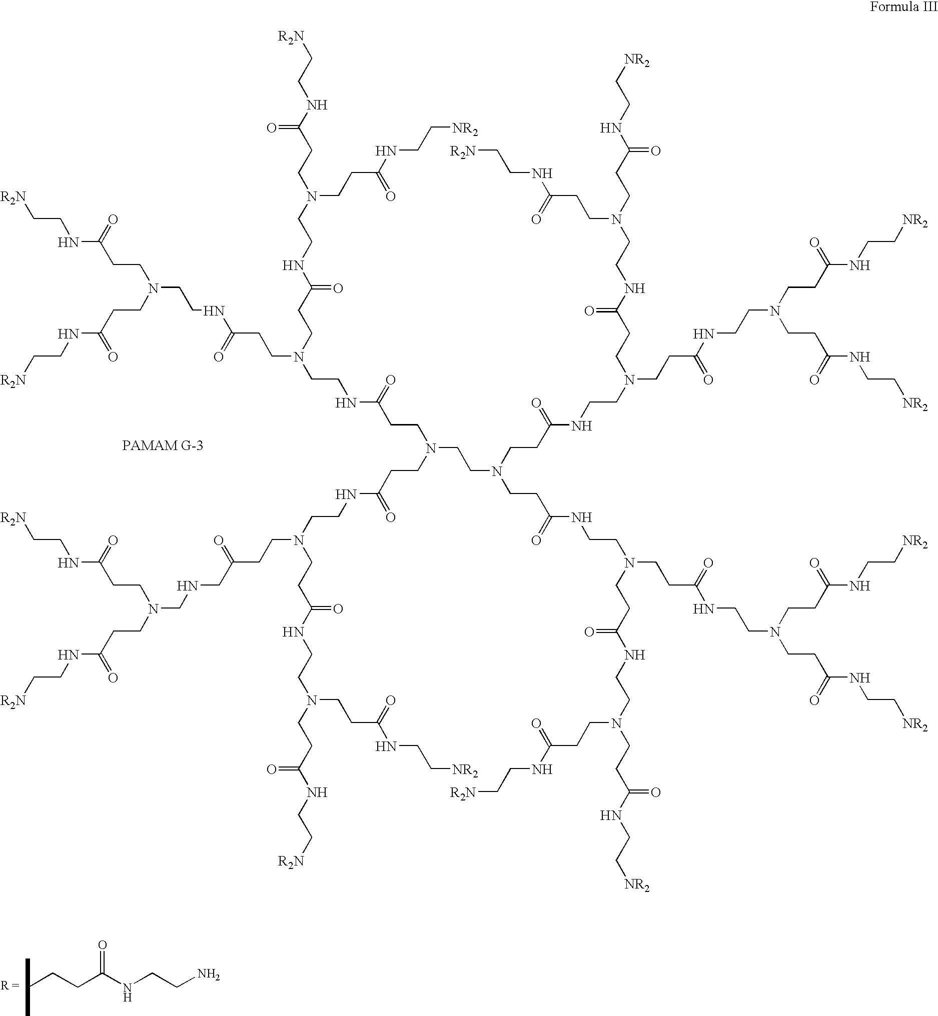

In some embodiments, the conjugate is a nanoparticle. In some embodiments, the particle size of the nanoparticle is in the range of about 10 nm to about 800 nm. In some embodiments, the particle size of the nanoparticle is in the range of about 10 nm to about 700 nm; about 10 nm to about 600 nm; about 10 nm to about 500 nm; about 10 nm to about 400 nm; about 10 nm to about 300 nm; about 10 mu to about 250 nm; about 10 nm to about 200 nm; about 10 nm to about 100 nm; about 10 nm to about 50 nm; about 50 nm to about 700 nm; about 50 nm to about 600 nm; about 50 nm to about 500 nm; about 50 nm to about 400 nm; about 50 nm to about 300 nm; about 50 nm to about 250 nm; about 50 nm to about 200 nm; about 50 nm to about 100 nm; about 100 nm to about 700 nm; about 100 nm to about 600 nm; about 100 nm to about 500 nm; about 100 nm to about 400 nm; about 100 nm to about 300 nm; about 100 nm to about 250 nm; about 100 nm to about 200 nm; about 200 nm to about 600 nm; about 200 nm to about 500 nm; about 200 nm to about 300 nm; about 200 nm to about 250 nm; about 300 nm to about 700 nm; about 300 nm to about 600 nm; about 300 nm to about 500 mu; about 300 nm to about 400 nm; about 400 nm to about 700 nm; about 400 nm to about 600 nm; about 400 nm to about 500 nm; about 500 nm to about 800 nm; about 500 mu to about 700 nm; about 500 nm to about 600 nm; about 600 nm to about 800 nm; about 600 nm to about 700 nm; or about 700 nm to about 800 mu. In some embodiments, the particle size of the nanoparticle is such that it is permeable through a cell-membrane.

In some embodiments, the conjugate is soluble in aqueous medium. In some embodiments, the conjugation of the perfluorocarbon compound with the cationic polymer results in enhanced solubility (up to 10 g/mL in water depending on the properties of cationic polymers) in aqueous medium as compared to an unconjugated perfluocarbon compound. The enhanced solubility of the perfluorocarbon compound may increase the ability of the perfluorocarbon compound to sustain life over longer periods resulting in a longer dose dependent circulatory half-life.

In some embodiments, the conjugate is administered to a mammal. In some embodiments, the conjugate is administered to a human. In some embodiments, the conjugate supplements the blood of a mammal.

In some embodiments, the conjugate transports oxygen with a p50 of about 10 mmHg to about 50 mmHg. In some embodiments, the conjugate transports oxygen with a p50 of about 10 mmHg to about 40 mmHg; about 10 mmHg to about 30 mmHg; about 10 mmHg to about 20 mmHg; about 15 mmHg to about 45 mmHg; about 15 mmHg to about 40 mmHg; about 15 mmHg to about 30 mmHg; about 15 mmHg to about 20 mmHg; about 20 mmHg to about 45 mmHg; about 20 mmHg to about 40 mmHg; about 20 mmHg to about 35 mmHg; about 20 mmHg to about 25 mmHg; about 25 mmHg to about 50 mmHg; about 25 mmHg to about 40 mmHg; about 25 mmHg to about 35 mmHg; about 30 mmHg to about 45 mmHg; about 35 mmHg to about 50 mmHg; about 35 mmHg to about 45 mmHg; or about 40 mmHg to about 45 mmHg.

In some embodiments, the conjugate transports oxygen of about 10% to 50% by volume. In some embodiments, the conjugate transports oxygen of about 10% to 40% by volume; about 10% to 30% by volume; about 10% to 20% by volume; about 15% to 40% by volume; about 15% to 35% by volume; about 15% to 30% by volume; about 15% to 25% by volume; about 15% to 20% by volume; about 20% to 45% by volume; about 20% to 35% by volume; about 20% to 30% by volume; about 25% to 40% by volume; about 25% to 35% by volume; about 30% to 40% by volume; about 35% to 45% by volume; about 35% to 40% by volume; about 40% to 50% by volume; or about 40% to 45% by volume. In some embodiments, the conjugation of the perfluorocarbon compound with the cationic polymer results in enhanced oxygen absorbing ability as compared to an unconjugated perfluocarbon compound.

### B. Methods of Use

In one aspect, there is provided a method of supplementing blood of a mammal comprising administering to the mammal a composition comprising a conjugate of a perfluorocarbon compound and a cationic polymer and a pharmaceutically acceptable carrier, wherein the conjugate is a blood substitute. In some embodiments, the mammal needs blood transfusion. In some embodiments, the mammal is in trauma or has recently experienced trauma. The mammal in trauma can be a mammal in need of transfusion. Other examples of mammal in trauma include, but are not limited to, injury, stroke, hemorrhage, bleeding, wound, etc.

“Mammal” or its grammatical equivalents and “patient” are used interchangeably herein and refer to a warm-blooded animal. In some embodiments, the “mammal” refers to a human. The mammal can also be an animal, such as but not limited to, domestic animals (e.g., dogs, cats and the like), farm animals (e.g., cows, sheep, pigs, horses and the like) and laboratory animals (e.g., rats, mice, guinea pigs and the like).

The composition comprising the conjugate and a pharmaceutically acceptable excipient may be administered to recipients, for example, by infusion, by intravenous or intra-arterial injection, or by other means. In some embodiments, the administration is by an implant, injection or transfusion. The compositions can be used as substitutes for red blood cells in any application where red blood cells are used. In some embodiments, the compositions can be used for the treatment of hemorrhage where blood volume is lost. In some embodiments, the compositions can be used as replacement for blood during surgical procedures where the patient's blood is removed and saved for reinfusion at the end of surgery or during recovery (e.g., acute normovolemic hemodilution or hemoaugmentation, etc.).

In some embodiments, the dose of the composition can be from 10 mg to 5 grams or more of conjugate per kilogram of patient body weight. Thus, a dose of conjugate for a human patient can be from a few grams to over 350 grams. It will be appreciated that the unit content of active ingredients contained in an individual dose of each dosage form need not in itself constitute an effective amount since the necessary effective amount could be reached by administration of a plurality of administrations as injections, etc. The selection of dosage depends upon the dosage form utilized, the condition being treated, and the particular purpose to be achieved according to the determination of the ordinarily skilled artisan in the field.

The administration of the composition can occur for a period of seconds to hours depending on the purpose of the usage. For example, in a blood delivery vehicle, the usual time course of administration is as rapid as possible. In some embodiments, the infusion rates for compositions as blood replacements can be from about 10 mL to about 1000 mL/hour; 10 mL to about 500 mL/hour; 10 mL to about 200 mL/hour; about 50 mL to about 1000 mL/hour; about 50 mL to about 800 mL/hour; about 50 mL to about 500 mL/hour; about 50 mL to about 200 mL/hour; about 50 mL to about 100 mL/hour; about 100 mL to about 1000 mL/hour; about 100 mL to about 800 mL/hour; about 100 mL to about 500 mL/hour; about 100 mL to about 200 mL/hour; about 250 mL to about 1000 mL/hour; about 250 mL to about 500 mL/hour; about 250 mL to about 300 mL/hour; about 400 mL to about 800 mL/hour; about 400 mL to about 500 mL/hour; about 500 mL to about 1000 mL/hour; about 500 mL to about 800 mL/hour; about 500 mL to about 600 mL/hour; about 600 mL to about 800 mL/hour; about 600 mL to about 700 mL/hour; about 800 mL to about 1000 mL/hour; or about 800 mL to about 900 mL/hour. In some embodiments, the infusion rates for compositions can be about 1 mL/kg/hour to about 300 mL/kg/hour, 1 mL/kg/hour to about 100 mL/kg/hour, 1 mL/kg/hour to about 50 mL/kg/hour, or from about 1 mL/kg/hour to about 25 mL/kg/hour.

In some embodiments, the mammal treated using the composition suffers from anemia, hypoxia or ischemia.

In some embodiments, the compositions can be used to treat anemia by providing additional oxygen carrying capacity in a patient that is suffering from anemia and/or by stimulating hematopoiesis.

The compositions provided herein can pass through the narrowed and/or obstructed or partially obstructed blood vessel due to its small size and high solubility. The compositions can be used to deliver oxygen to areas that red blood cells cannot penetrate. These areas can include any tissue areas that are located downstream of obstructions to red blood cell flow, such as but not limited to, areas downstream of thrombi, sickle cell occlusions, arterial occlusions, angioplasty balloons, surgical instrumentation, tissues that are suffering from oxygen starvation or are hypoxic, and the like. In some embodiments, the composition is used in treating ischemia and hypoxia. Ischemia can be due to various reasons including, but not limited to, heart disease, myocardial stunning and hibernation, acute or unstable angina, emerging angina, infarct, transient ischemic attack, cerebrovascular accident, ischemia in brain tissue, for example due to stroke or head injury, ruptured arteriovenous malformation, and peripheral artery occlusive disease. The heart, the kidneys, and the brain are among the organs that are sensitive to inadequate blood supply. In some embodiments, the compositions can be used to provide additional oxygen carrying capacity to an individual (such as an athlete, soldier, mountaineer, aviator, smoke victim, etc.) desiring such additional oxygen carrying capacity.

The recovery of tissues from physical damage such as burns can also be accelerated by pretreatment with the compositions provide herein, which can allow increased perfusion and oxygenation of the tissues.

In some embodiments, the compositions may be used for the treatment of sickle cell anemia patients. Sickle cell anemia patients in vasoocclusive crisis can be treated by transfusion of the compositions provided herein in conjunction with dilution and pain management. The compositions provided herein may not only deliver oxygen thereby preventing further sickling (as do red blood cells), they may also penetrate vessels already occluded with deformed red cells to better alleviate pain and minimize tissue damage. It is contemplated that the compositions provided herein offer a therapeutic advantage in treatment of sickle cell anemia patients, since they may elicit a lesser degree of vasoconstriction or none at all. The compositions provided herein may be used in place of packed red cells for preoperative transfusion of sickle cell anemia patients to minimize the risk of anesthesia. The compositions provided herein may also be administered periodically to minimize the risk of stroke.

In some embodiments, the compositions provided herein may be used in events such as, but not limited to, surgery, injury with bleeding, gastrointestinal hemorrhage and diffuse hemorrhagic disorders of various types. The compositions provided herein may be used in military battles where the blood transfusions are required in the battlefield.

In some embodiments, the compositions provided herein may be used in cardiac surgery, such as, but not limited to, cardiopulmonary bypass.

In some embodiments, the compositions provided herein may be used in assisting respiration in a mammal having a lung disorder, wherein lung surfactant or lung flexibility is inadequate to allow normal respiration, so that the mammal can breathe normally using ambient gas without mechanical assistance. Some examples of the lung disorder include, but are not limited to, respiratory distress syndrome (RDS), lung surfactant deficiency, emphysema, hyperinflated lung syndrome, or other types of lung injury or deterioration.

In some embodiments, the compositions provided herein may be used for reducing the body burden of a blood borne infection in a patient. The example of blood borne infection includes, but is not limited to, AIDS virus. The emergency replacement procedure or method for rapid and drastic reduction of the body burden of AIDS virus residing primarily in the formed elements of the blood can involve the removal of all blood from the patient and replacement with a blood substitute (in physiological saline or equivalent isosmotic) in order to attempt a “scrubbing” in totality of the AIDS containing blood from all of the vital organs and then replacement of the blood substitute with whole blood of the same type as the patient. The compositions provided herein can be used as the blood substitute in the partial or complete blood replacement procedure. It is contemplated that the replacement procedure would be effective in reducing the body burden of any toxicant, contaminant or product of disease (e.g., leukemia, blood poisoning infection, aberrant enzyme, etc.) that is primarily blood born and exerts its major toxicity from that compartment of the body.

In one embodiment, the compositions provided herein can be administered to non-human animal. Although humans have four main red cell antigens (A, B, O and Rh), accounting for 12 main blood types, non-human animals exhibit far greater blood type diversity. The existence of larger numbers of blood types can complicate the use of donated blood in non-human animal transfusions. The compositions provided herein, which can be used regardless of the blood type of the recipient, thus finds additional utility as a blood substitute for non-human animals (e.g., dogs, horses, cats, etc.).

The compositions provided herein can be delivered by implantable delivery devices (such as cartridges, implants, etc.) that contain the compositions, and that are capable of releasing the compositions into the circulation in response to a sensed need for increased oxygen carrying capacity. In some embodiments, such devices can deliver the compositions at a constant rate. In some embodiments, the devices can be controlled by sensing means (such as electronic probes of O₂ level, CO₂ level, etc.) so as to deliver the composition at a rate commensurate with the patient's oxygen carrying capacity needs. Such sensing means may themselves be implantable, or part of the implanted device, or may be located extracorporeally.

The conjugate provided herein may also be used to form non-pharmaceutical compositions that can be used, for example, as reference standards for analytical instrumentation needing such reference standards, reagent solutions, control of gas content of cell cultures, for example by in vitro delivery of oxygen to a cell culture, and removal of oxygen from solutions. The compositions provided herein can be used to remove oxygen from solutions requiring the removal of oxygen, and as reference standards for analytical assays and instrumentation. The compositions provided herein can also be used in vitro to enhance cell growth in cell culture by maintaining oxygen levels.

Besides the blood substitute for mammals, the compositions provided herein can be used as a perfusate for preservation of the internal organs. The compositions provided herein may be used to oxygenate donated tissues and organs during transport.

### C. Process of Preparation

In one aspect, there is provided a process of preparing a conjugate of a perfluorocarbon compound and a cationic polymer wherein the conjugate is a blood substitute, the process comprising reacting the perfluorocarbon compound with the cationic polymer. In another embodiment, there is provided a method of making a conjugate of a perfluorocarbon compound and a cationic polymer wherein the conjugate is a blood substitute, the method comprising reacting the perfluorocarbon compound with the cationic polymer thereby resulting in the conjugate.

The reaction is carried out in presence of a base. In some embodiments, the base is an organic or an inorganic base. The organic or inorganic bases are well known in the art. Without being bound by any theory, the examples of the bases include, but are not limited to, hydroxide such as, sodium hydroxide or potassium hydroxide; carbonate such as, sodium carbonate or sodium bicarbonate; sodium acetate; ammonia; tertiary amine such as, triethylamine or dimethylethanolamine; silicate such as, silicate of sodium or potassium etc. In some embodiments, the base is selected from the group consisting of a tertiary amine, a carbonate, or a silicate of sodium or potassium. The reaction can be carried out in the presence of an organic solvent. Organic solvents are well known in the art. Examples of the solvent include, but are not limited to, methylethylketone, dichloromethane, carbon tetrachloride, ethyl acetate, acetonitrile, etc. The reaction can be carried out at room temperature or can be heated depending on the reactants. It is well within the skill of the person of ordinary skill in the art to choose a solvent, a base, and the reaction conditions based on the perfluorocarbon compound and the cationic polymer that are undergoing the reaction.

The process of reacting the perfluorocarbon compound and the cationic polymer may include maintaining the steps of the process under conditions sufficient to minimize microbial growth or bioburden, such as conducting the reaction in an inert atmosphere or in sterile or aseptic conditions.

The perfluororcarbon compound may be available commercially. Alternatively, the compound can be produced according to the processes well known in the art. For example, the organic compound can be perfluorinated to remove all hydrogens and unsaturated by a multiple stage fluorination technique. The organic compound can be first subjected to fluorination using a CoF₃ particulate bed operated at a temperature of approximately 275°-427° C. The chemical composition can then be carried through the bed with a nitrogen carrier gas at a pressure from ambient up to 2 psi at a nitrogen to organic ratio in the range of 10/90 to 90/10. Yields from this fluorination can be 50 to 80% of theoretical.

The cationic polymer may be available commercially. Alternatively, the cationic polymer can be produced according to processes well known in the art. An example of a process to prepare the cationic polymer of Formula II is as shown in Example 1.

For quality control, the process can be profiled by TLC analysis, LC-MS/MS (liquid chromatography-mass spectrometry) method or other analytic methods. Various separation/analysis techniques are well known in the art. For example, nuclear magnetic resonance (NMR), infrared spectroscopy (IR), UV-V is (ultraviolet-visible), mass spectrometry (MS), conductometric titrations, etc. can be used as analytical methods for identification of the products during and after the reaction.

### D. Pharmaceutical Formulations and Routes of Administration

In one aspect, there is provided a pharmaceutical composition comprising a conjugate comprising a perfluorocarbon compound and a cationic polymer wherein the conjugate is a blood substitute, and a pharmaceutically acceptable carrier. “Pharmaceutically acceptable carrier” as used herein, means those carriers which retain the biological effectiveness and properties of the compositions provided herein, and which are not biologically or otherwise undesirable.

The compositions provided herein may be incorporated in conventional pharmaceutical formulations (e.g., injectable solutions) for use in treating mammals in need thereof. Pharmaceutical compositions can be administered by subcutaneous, intravenous, or intramuscular injection, or as large volume parenteral solutions and the like.

The compositions provided herein can be formulated into blood substitute formulations. For example, a parenteral composition can comprise a sterile isotonic saline solution. The composition can be either in a form suitable for direct administration, or in a concentrated form requiring dilution prior to administration. The composition can contain between 0.001% and 90% (w/v) of the conjugate. In some embodiments, the composition may contain from about 5 percent to about 90 percent; from about 5 percent to about 80 percent; from about 5 percent to about 70 percent; from about 5 percent to about 60 percent; from about 5 percent to about 50 percent; from about 5 percent to about 40 percent; from about 5 percent to about 30 percent; from about 5 percent to about 20 percent; from about 5 percent to about 15 percent; from about 5 percent to about 10 percent; from about 10 percent to about 80 percent; from about 10 percent to about 70 percent; from about 10 percent to about 60 percent; from about 10 percent to about 50 percent; from about 10 percent to about 40 percent; from about 10 percent to about 30 percent; from about 10 percent to about 20 percent; from about 20 percent to about 80 percent; from about 20 percent to about 70 percent; from about 20 percent to about 60 percent; from about 20 percent to about 50 percent; from about 20 percent to about 40 percent; from about 20 percent to about 30 percent; from about 30 percent to about 80 percent; from about 30 percent to about 70 percent; from about 30 percent to about 60 percent; from about 30 percent to about 50 percent; from about 30 percent to about 40 percent; from about 40 percent to about 80 percent; from about 40 percent to about 70 percent; from about 40 percent to about 60 percent; from about 40 percent to about 50 percent; from about 50 percent to about 80 percent; from about 50 percent to about 70 percent; or from about 50 percent to about 60 percent conjugate in solution (% weight per volume). The selection of percent conjugate depends at least in part on the osmotic properties of the composition and the desired osmotic pressure for each indication.

A dose of the composition can be from about 1 mg to about 15 grams of conjugate per kilogram of patient body weight. When used as an oxygen carrying composition, or as a blood substitute, the dosage may range between 100 to 7500 mg/kg patient body weight, 100 to 6500 mg/kg patient body weight, 100 to 5500 mg/kg patient body weight, 100 to 4500 mg/kg patient body weight, 100 to 3500 mg/kg patient body weight, 100 to 2500 mg/kg patient body weight, 100 to 2000 mg/kg patient body weight, 100 to 1000 mg/kg patient body weight, 100 to 900 mg/kg patient body weight, 100 to 800 mg/kg patient body weight, 100 to 500 mg/kg patient body weight, 500 to 7000 mg/kg body weight, 500 to 6000 mg/kg body weight, 500 to 5000 mg/kg body weight, 500 to 1000 mg/kg body weight, 1000 to 7000 mg/kg body weight, 1000 to 6000 mg/kg body weight, 1000 to 5000 mg/kg body weight, 1000 to 4000 mg/kg body weight, 1000 to 3000 mg/kg body weight, 1000 to 2000 mg/kg body weight, 2000 to 7000 mg/kg body weight; 2000 to 6000 mg/kg body weight, 2000 to 5000 mg/kg body weight, or 2000 to 4000 mg/kg body weight. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The compositions provided herein may comprise a physiologically compatible electrolyte vehicle isosmotic with whole blood. The physiologically acceptable solution can be, but is not limited to, physiological saline, a saline-glucose mixture, Ringer's solution, lactated Ringer's solution, Locke-Ringer's solution, Krebs-Ringer's solution, Hartmann's balanced saline, heparinized sodium citrate-citric acid-dextrose solution, and polymeric plasma substitutes, such as, but is not limited to, polyethylene oxide, polyvinyl pyrrolidone, polyvinyl alcohol and ethylene oxide-propylene glycol condensates. In some embodiments, the composition comprises from about 0.1 to about 10% of the physiologically acceptable solution.

The compositions provided herein comprise inert constituents including pharmaceutically-acceptable carriers, diluents, fillers, salts, and other materials well-known in the art, the selection of which depends on the dosage form utilized, the condition being treated, the particular purpose to be achieved according to the determination of the ordinarily skilled artisan in the field and the properties of such additives. For example, the compositions provided herein may include, but are not limited to, one or more of 0-200 mM of one or more physiological buffers (e.g., sodium gluconate, acetate, phosphate, citrate, bicarbonate, or good's buffer), 0-200 mM of one or more carbohydrates (e.g., reducing carbohydrates such as glucose, maltose, lactose or non-reducing carbohydrates such as sucrose, trehalose, raffinose, mannitol, isosucrose or stachyose), 0-200 mM of one or more alcohols or poly alcohols (such as polyethylene glycols, propylene glycols, dextrans, or polyols), 0-200 mM of one or more physiologically acceptable salts (e.g., sodium chloride, potassium chloride, sodium acetate, calcium chloride, magnesium chloride), and 0-1% of one or more surfactants (e.g., Tween™ (polysorbate 80)), and/or 0-20 mM of N-acetyl cysteine.

The compositions provided herein may also contain one or more surfactant and 0-200 mM of one or more chelating agent (for example, ethylenediamine tetraacetic acid (EDTA), ethylene glycol-bis (beta-aminoethyl ether) N,N,N′,N′-tetraacetic acid (EGTA), ophenanthroline, diethylamine triamine pentaacetic acid (DTPA also known as pentaacetic acid) and the like). The surfactant can be 0.005-1% of the composition. The compositions can be at pH of about 6.5-9.5. In some embodiments, the composition may contain 0-150 mM NaCl, 0-10 mM sodium phosphate, 0.01-0.1% surfactant, and/or 0-50 μM of one or more chelating agents at pH 6.6-7.8.

The compositions provided herein may contain physiologically acceptable crystalloids or chemical components which are present in normal blood capable of increasing the osmolarity of the blood substitute. The physiologically acceptable crystalloid components are those that are present in normal blood and their concentrations are similar to those of normal blood. Physiologically acceptable crystalloid components can be inorganic ions and organic components which are present in normal blood. The amino acid component of the physiologic crystalloid component can be any of the naturally occurring amino acids and includes, but is not limited to, alanine, arginine, asparagine, aspartic acid, cysteine, cystine, 3,5-dibromotyrosine, 3,5-diiodotyrosine, glutamic acid, glutamine, glycine, histidine, hydroxylysine, dydroxyproline, isoleucine, leucine, lysine, methionine, phenylalanine, proline, serine, threonine, thyroxine, tryptophane, tyrosine and valine. It is to be understood that by naturally occurring amino acid it is meant that the above amino acids have a proper stereochemical and optical chemical configurations. Of the above amino acids, arginine, histidine isoleucine, leucine, lysine, methionine, phenylalanine, threonine, tryptophane and valine are all known to be essential amino acids.

Methods to prepare the composition include, for example, simple mixing, sequential addition, emulsification, diafiltration and the like.

The compositions provided herein may be presented as a kit, a pack or dispenser device containing one or more unit dosage forms containing the active ingredient. The kit, pack or dispenser device may be accompanied by instructions for administration.

The compositions provided herein may be stored in oxygen impermeable containers (for example, stainless steel tanks, oxygen impermeable plastic bags, or plastic bags overwrapped with low oxygen permeably plastic bags wherein an oxygen scavenger is placed between the internal plastic bag and the overwrapped plastic bag). In some embodiments, the composition is stored in the absence of oxygen. The composition may be oxygenated prior to use such as, by way of example only, oxygenating before using in the catheter for cardiac therapy. In some embodiments, the composition can be stored in oxygen permeable or oxygen impermeable (“anoxic”) containers in an oxygen controlled environment. Such oxygen controlled environments can include, for example, glove boxes, glove bags, incubators and the like. The oxygen content of the oxygen controlled environment may be low relative to atmospheric oxygen concentrations. In some embodiments, the composition can be packaged in sealed Tyvek or Mylar (polyethylene terephthalate) bags or pouches. In some embodiments, the composition can be lyophilized and stored as a powder. The preparations may be stored at room or elevated temperature or under refrigeration.

The container for storing the composition can be manufactured from a variety of materials, including polymer films, (e.g., an essentially oxygen-impermeable polyester, ethylene vinyl alcohol (EVOH), or nylon), and laminates thereof, such as a transparent laminate (e.g., a silicon oxide or EVOH containing-laminate) or a metal foil laminate (e.g., a silver or aluminum foil laminate). The polymer can be a variety of polymeric materials including, but not limited to, a polyester layer (e.g., a 48 gauge polyester), nylon or a polyolefin layer, such as polyethylene, ethylene vinyl acetate, or polypropylene or copolymers thereof.

The container can be of a variety of constructions, including, but not limited to, vials, cylinders, boxes, etc. In some embodiments, the container is in the form of a bag. A suitable bag can be formed by continuously bonding one or more (e.g., two) sheets at the perimeter(s) thereof to form a tightly closed, oxygen impermeable, construction having a fillable center. In the case of laminates comprising polyolefins, such as linear low density, low density, medium or high density polyethylene or polypropylene and copolymers thereof, the perimeter of the bag may be bonded or sealed using heat. It is well within the skill of the art to determine the shape of the bag and the appropriate temperature to generate a tightly closed, oxygen and/or moisture impermeable construction. Where the container is a film, such as a polyester film, the film can be rendered essentially oxygen-impermeable by a variety of suitable methods. The film can be laminated or otherwise treated to reduce or eliminate the oxygen permeability.

In some embodiments, one or more antioxidants, such as, but not limited to, ascorbate, gluathione, acetylcsyteine, methionine, tocopherol, butyl hydroxy toluene, or butyl hydroxy anisole may be added to further stabilize the preparation. In some embodiments, the composition of such storage containers may be subjected to irradiation or other sterilization treatment sufficient to extend the shelf-life of the compositions.

The composition may be stored at suitable storage temperatures when stored in a low oxygen environment. Suitable storage temperature for storage of one year or more is between about 0° C. and about 40° C.; about 0° C. and about 35° C.; about 0° C. and about 30° C.; about 0° C. and about 25° C.; and 0° C. and about 20° C.; about 0° C. and about 10° C.; about 0° C. and about 5° C.; about 10° C. and about 25° C.; about 10° C. and about 20° C.; about 20° C. and about 40° C; or about 20° C and about 30° C.

### EXAMPLES

The present technology is further understood by reference to the following examples, which are intended to be illustrative. The present technology is not limited in scope by the Examples. Any methods that are functionally equivalent are within the scope of the present technology. Various modifications of the present technology in addition to those described herein will become apparent to those skilled in the art from the foregoing description and accompanying figures. Such modifications fall within the scope of the claims.

In these examples and elsewhere, abbreviations have the following meanings:

| | |
|---|---|
| IR | infrared |
| g | gram |
| GPC | gel permeation chromatography |
| h | hour(s) |
| HCL | hydrochloride |
| ¹HNMR | proton nuclear magnetic resonance |
| Kg | kilogram |
| μM | micromolar |
| mg | milligram |
| mL | milliliter |
| mM | millimolar |
| MS | mass spectroscopy |
| NaCl | Sodium chloride |
| NaOH | sodium hydroxide |
| NMR | nuclear magnetic resonance |
| PG | polyglycidol |
| PEG | polyethylene glycol |
| MPEG | poly(ethyleneglycol) monomethylether |
| THF | tetrahydrofuran |
| w/w | weight/weight |

### Example 1

### Preparation of the Amine-Modified Polyethylene-Glycol Based Cationic Hyperbranched Polymer of Formula I

### Step 1: Synthesis of Copolymer of Hyperbranched Polyglycidol (PG) and Polyethylene Glycol (PEG):

Tris(hydroxymethyl)propane (120 mg, Fluka) is stirred with 0.2 mL potassium methylate (Fluka) solution and excess methanol is removed under vacuo at about 50° C. Glycidol (5 mL, can be bought commercially or purified by vacuum distillation and stored in the refrigerator) is added dropwise at about 95° C. over about 10 h. After the addition of glycidol, the mixture is stirred for additional about 2 h, after which 20 mL MPEG-epoxide (Epoxide terminated poly(ethyleneglycol) monomethylether, synthesized by reaction of MPEG(Mn-350), sodium hydroxide and epichlorohydrin, see U.S. Pat. No. 6,221,977) is added dropwise over about 12 h. The mixture may be stirred for an additional about 3 h. The viscous polymer is dissolved in methanol and passed though cation exchange resin to remove potassium ions. Polymer is precipitated twice from diethyl ether to remove unreacted PEG-epoxide and subsequently dried at 70° C. in vacuo. The product can be analysed by NMR. GPC analysis (Gel Permeation Chromatography) of the non-dialysed product-Mn-53000, Mw/Mn-6.01.

### Step 2: Synthesis of Amine Terminated PG-PEG:

PG-PEG of step 1 (16 g) is dissolved in about 100 mL THF and is reacted with methane sulfonyl chloride (about 1.17 mL, ˜20% of OH groups) in presence of triethylamine (3 mL) for 12 h. The salt is filtered off and polymer is isolated by precipitation in ether. The dried polymer is dissolved in 100 mL dioxane to which 40 mL tris(2-aminoethyl)amine is added and refluxed for 24 h. Dioxane is removed by rotary evaporation. Solid then is dissolved in a minimum amount of methanol and the polymer is twice precipitated in diethyl ether. The obtained polymer (15 g) is dissolved in 100 mL water and added to a stirred solution of formic acid (90% w/w) and formaldehyde ( 37% w/w)(15 each) at 0° C. The reaction mixture is refluxed at 95° C. overnight. The volatiles is removed in vacuo. Polymer is extracted with dichloromethane after adjusting the pH of the aqueous solution to 10 with sodium hydroxide. Finally, the polymer is purified by dialysis using regenerated cellulose acetate membrane (MWCO 1000). The product is characterized by ¹HNMR, GPC analysis and conductometric titrations. A conductometric titration method may include using HCl and NaOH. The GPC analysis of the non-dialysed final product-Mn-116700, Mw/Mn-1.72 Rg-24.5 nm.

### Step 3: Synthesis of Quaternized Amines:

The tertiary amine groups in PG-PEG amine can be quaternized using ethyl bromide. PG-PEG amine (1.2 g) is dissolved in acetonitrile (16 mL) and methanol (8 mL) and ethyl bromide (150 mg) is added. The solution is refluxed over night and the solvent is removed in a rotary evaporator. The final product is dissolved in water and freeze dried and is characterized by ¹HNMR and conductometric titrations.

### Example 2

### Reaction of Perfluorocarbon Compound with the Cationic Polymer

A mixture of the cationic polymer of Example 1, perfluorocarbon compound, potassium carbonate, and methylethylketone is stirred at 35°-45° C. for 5-10 h. The solution is cooled, filtered and the solvent is removed using a rotary evaporator. There remains a resinous material which is analyzed using NMR, IR and MS. The fluorine content of the material is calculated using elemental analysis or MS.

While preferred embodiments of the present technology have been shown and described herein, it will be obvious to those skilled in the art that such embodiments are provided by way of example only. Numerous variations, changes, and substitutions will now occur to those skilled in the art without departing from the present technology. It should be understood that various alternatives to the embodiments of the present technology described herein may be employed in practicing the present technology. It is intended that the following claims define the scope of the present technology and that methods and structures within the scope of these claims and their equivalents be covered thereby.

### EQUIVALENTS

The present disclosure is not to be limited in terms of the particular embodiments described in this application, which are intended as single illustrations of individual aspects of. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A conjugate comprising a perfluorocarbon compound and a cationic polymer wherein said conjugate is a blood substitute.

2. The conjugate of claim 1, wherein said perfluorocarbon compound is an oxygen-transferable saturated perfluorocarbon.

3. The conjugate of claim 1, wherein said perfluorocarbon compound is having 8 to 25 carbon atoms.

4. The conjugate of claim 1, wherein said perfluorocarbon compound is perfluorodecalin, perfluoro(methyldecalin), perfluorooctyl bromide, or dodecafluoropentane.

5. The conjugate of claim 1, wherein said perfluorocarbon compound is a perfluorocycloalkane or a perfluoro(alkylcycloalkane).

6. The conjugate of claim 5, wherein said perfluoro(alkylcycloalkane) is selected from the group consisting of perfluoro(methylpropylcyclohexanes), perfluoro(butylcyclohexanes), perfluoro(trimethylcyclohexanes), perfluoro(ethylpropylcyclohexanes) and perfluoro (pentylcyclohexanes).

7. The conjugate of claim 1, wherein said perfluorocarbon compound is a perfluoro(alkyltetrahydropyrans).

8. The conjugate of claim 7, wherein said perfluoro(alkyltetrahydropyrans) is selected from the group consisting of perfluoro(butyltetrahydropyrans), perfluoro(pentyltetrahydropyrans) and perfluoro(hexyltetrahydropyrans).

9. The conjugate of claim 1, wherein said perfluorocarbon compound is a perfluoro(alkyltetrahydrofurans).

10. The conjugate of claim 9, wherein said perfluoro(alkyltetrahydrofurans) is selected from the group consisting of perfluoro(pentyltetrahydrofurans), perfluoro(hexyltetrahydrofurans) and perfluoro(heptyltetrahydrofurans).

11. The conjugate of claim 1, wherein said perfluorocarbon compound is a perfluoro(N-alkylpiperidines).

12. The conjugate of claim 11, wherein said perfluoro(N-alkylpiperidines) is selected from the group consisting of perfluoro(N-pentylpiperidines), perfluoro(N-hexylpiperidines) and perfluoro (N-butylpiperidine).

13. The conjugate of claim 1, wherein said perfluorocarbon compound is a perfluoro(N-alkylmorpholines).

14. The conjugate of claim 1, wherein said perfluoro(N-alkylmorpholines) is selected from the group consisting of perfluoro(N-pentylmorpholines), perfluoro(N-hexylmorpholines) and perfluoro(N-heptylmorpholines).

15. The conjugate of claim 1, wherein said perfluorocarbon compound is a perfluoro(tert-amine).

16. The conjugate of claim 15, wherein said perfluoro(tert-amine) is selected from the group consisting of perfluoro(diethylhexylamines), perfluoro(dipropylbutylamines) and perfluoro (diethylcyclohexyl amines).

17. The conjugate of claim 1, wherein said perfluorocarbon compound is a perfluoro(dioxa-alkane).

18. The conjugate of claim 17, wherein said perfluoro(dioxa-alkane) is selected from the group consisting of perfluoro(tetramethylene glycol diisoproyl ether), perfluoro(trimethylene glycol diisopropyl ether), perfluoro(trimethylene glycol diisobutyl ether), and perfluoro(isopropylidene glycol di-n-propyl ether).

19. The conjugate of claim 1, wherein said conjugate is a nanoparticle.

20. The conjugate of claim 1, wherein a particle size of said conjugate is 10 nm to 800 nm.

21. The conjugate of claim 1, wherein a particle size of said conjugate is 50 nm to 250 nm.

22. The conjugate of claim 1, wherein said conjugate is soluble in aqueous medium.

23. The conjugate of claim 1, wherein said conjugate is administered to a mammal.

24. The conjugate of claim 1, wherein said cationic polymer is a polyethylene-glycol based cationic hyperbranched polymer.

25. The conjugate of claim 1, wherein said cationic polymer is an amine-modified polyethylene-glycol based cationic hyperbranched polymer.

26. The conjugate of claim 1, wherein said conjugate supplements blood of a mammal.

27. The conjugate of claim 1, wherein said conjugate transports oxygen with a p50 of about 10 mmHg to about 50 mmHg.

28. The conjugate of claim 1, wherein said conjugate transports oxygen of about 10% to 50% by volume.

29. The conjugate of claim 1, wherein said perfluorocarbon compound and said cationic polymer are conjugated by a covalent bond.

30. The conjugate of claim 1, wherein said conjugate has an enhanced solubility in aqueous medium as compared to an unconjugated perfluocarbon compound.

31. The conjugate of claim 1, wherein said conjugate has an enhanced oxygen absorbing ability as compared to an unconjugated perfluocarbon compound.

32. A pharmaceutical composition comprising said conjugate of claim 1 and a pharmaceutically acceptable carrier.

33. A container containing said pharmaceutical composition of claim 32.

34. A process of preparing a conjugate of a perfluorocarbon compound and a cationic polymer wherein said conjugate is a blood substitute, said process comprising reacting said perfluorocarbon compound with said cationic polymer in presence of a base.

35. The process of claim 34, wherein said base is an organic or an inorganic base.

36. The process of claim 34, wherein said base is selected from the group consisting of a tertiary amine, a carbonate, or a silicate of sodium or potassium.

37. A method of making a conjugate of a perfluorocarbon compound and a cationic polymer wherein said conjugate is a blood substitute comprising, reacting said perfluorocarbon compound with said cationic polymer thereby resulting in said conjugate.

38. The method of claim 37, wherein said reaction is carried out in presence of a base.

39. The method of claim 38, wherein said base is an organic or an inorganic base.

40. A method of supplementing blood of a mammal comprising administering to said mammal a composition comprising a conjugate of a perfluorocarbon compound and a cationic polymer wherein said conjugate is a blood substitute and a pharmaceutically acceptable carrier.

41. The method of claim 40, wherein said administering is by an implant, injection or transfusion.

42. The method of claim 40, wherein said mammal suffers from anemia, anemia related conditions, hypoxia or ischemia.

43. The method of claim 40, wherein said mammal needs a blood transfusion.

44. The method of claim 40, wherein said mammal is in trauma.
